(19) United States
(12) Patent Application Publication
(10) Pub. No.: US 2008/0193388 A1
(43) Pub. Date: Aug. 14, 2008

(54) Mouth wash gum

(76) Inventor: Patrick Anthony Tribulato, Omaha, NE (US)

Correspondence Address: Patrick Anthony Tribulato, 1922 South 61 St., Omaha, NE, 68106, US

(21) Appl. No.: 12/001,030

(22) Filed: Dec. 10, 2007

Related U.S. Application Data
(60) Provisional application No. 60/900,082, filed on Feb. 8, 2007.
(60) Provisional application No. 60/900,052, filed on Feb. 8, 2007.

(51) Int. Cl. A61K 8/18; A61Q 11/00

(52) U.S. Cl. 424/48

(57) ABSTRACT

A new and exciting way of delivering mouth wash to the mouth. The mouth wash is put into gum. The gum is chewed. The mouth wash is then delivered to the mouth through the gum as the gum is chewed. The gum has mouth wash in it. The mouth wash then comes out of the gum that is being chewed. The mouth wash then washes the mouth for dental hygiene.

### CROSS-REFERENCE TO RELATED APPLICATIONS

This Application has two provisional applications applied for with both of them being filled on Feb. 8, 2007. The first provisional application has the application No. 60/900,082 on the letter mailed to me on Mar. 5, 2007. The second provisional application has the No. 60/900,052 on the letter mailed to me on Apr. 18, 2007.

### FEDERALLY SPONSORED RESEARCH

Not Applicable

### SEQUENCE LISTING OR PROGRAM

Not Applicable

### BACKGROUND OF THE INVENTION

1. Field of Invention

This invention came about for the benefit it provided from health care for the teeth, gums and mouth. It also came about from the ease of use in chewing gum while at the same time providing health care to the teeth, gums and mouth. The mouth naturally produces saliva while you chew the gum and this action takes the antiseptic mouth wash all over the mouth. This is a great health benefit provided with very little effort.

2. Prior Art

The idea for this invention has not been thought of before. It is a simple idea to add antiseptic mouth wash to gum but no one has done it and it is new. There is no prior art for this invention and its concept is new and exiting. The original idea for this invention came about from chewing gum and the need to clean my teeth. Gum is a well known product and so is antiseptic mouth wash. They have been around for a long time. The idea of combining the two into one health care product is new and original to this invention.

The idea of providing health benefits of antiseptic mouth wash in gum is new and it is a good way of delivering the mouth wash.

### OBJECTS AND ADVANTAGES

The object of this invention is to provide health care to the teeth, gums and mouth. This is done be chewing the gum that has antiseptic mouth wash in it. The gum then releases the antiseptic mouth wash inside the mouth and helps provide health benefits of the mouth wash to the whole mouth. The advantages are:
(a) The most obvious is the health benefits provided to the mouth by the mouth wash. The teeth and gums are cleaned and teeth are strengthened. Also the antiseptic mouth wash fights against disease and tooth decay.
(b) The second benefit is that chewing gum is almost involuntary. The mouth is cleaned while you are anywhere doing anything. It takes little effort to chew gum and while you are chewing the gum you are cleaning your mouth.
(c) Another benefit is that you can give the gum to anyone who does not like to spend the time cleaning their teeth, gums and mouth. This is an effortless way of health care for the teeth, gums and mouth.

### SUMMARY

This is an invention that provides a health benefit for the teeth, gums and mouth. It is gum that has mouth wash in it. The gum is chewed and the antiseptic mouth wash cleans the teeth, gums and mouth.

### DRAWINGS

### Figures

The drawings show the gum, with antiseptic mouth wash in it, from two sides, from the front and the side.

### DETAILED DESCRIPTION

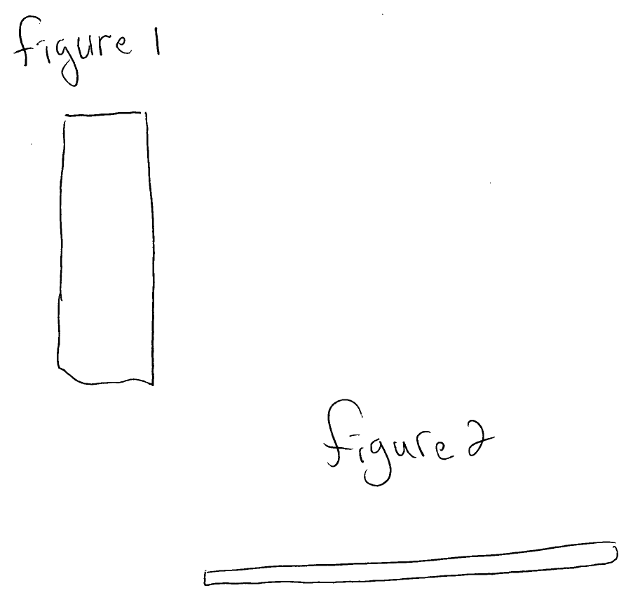

### FIGS. 1 and 2

FIG. 1 shows the front view of the gum with antiseptic mouth wash in it.

FIG. 2 shows the side view of the gum with the antiseptic mouth wash in it.

### OPERATIONS

### FIGS. 1 and 2

The gum is to be chewed in the mouth to release the antiseptic mouth wash to provide the health benefits of the antiseptic mouth wash to the teeth, gums and mouth.

### CONCLUSION

This is a good invention that provides a health benefit to the teeth, gums and mouth. The mouth is cleaned and the antiseptic mouth wash fights disease.

I claim:

1. A health care product comprising:
(1) Chewing gum that has antiseptic mouth wash in the gum
(2) The antiseptic mouth wash is flavored to make the gum more pleasant to chew
